(19) United States
(12) Patent Application Publication
(10) Pub. No.: US 2017/0202614 A1
(43) Pub. Date: Jul. 20, 2017

(54) CATHETER GUIDANCE AND PROCEDURE PLANNING

(71) Applicant: RAINBOW MEDICAL LTD., Herzliya (IL)

(72) Inventors: Yossi Gross, Moshav Mazor (IL); Yehuda Zadok, Kiryat Ono (IL)

(73) Assignee: RAINBOW MEDICAL LTD., Herzliya (IL)

(21) Appl. No.: 15/001,615

(22) Filed: Jan. 20, 2016

(51) Int. Cl. A61B 18/14; A61B 5/00; A61B 5/0215; A61B 5/06

(52) U.S. Cl. CPC A61B 18/1492; A61B 5/061; A61B 5/6852; A61B 5/0215; A61B 2018/00434

(57) ABSTRACT

A method includes, using a computer processor: (1) generating a map of vasculature of a subject, by: (a) displaying (i) a schematic representation of the vasculature including a blood vessel, and (ii) on the schematic representation, a first plurality of branch-site options; (b) receiving a user-selected first branch site, and responsively displaying (i) a schematic representation of a first branch of the vasculature that branches from the schematic representation of the blood vessel at the user-selected first branch site, and (ii) a second plurality of branch-site options; and (c) receiving a user-selected second branch site; (2) in response to receiving the user-selected second branch site, displaying the map; (3) detecting a physiological parameter of the subject; and (4) in response to the detected physiological parameter, determining a value for the parameter, and storing, in association with a location on the map, data representative of the value.

### FIELD OF THE INVENTION

Some applications of the present invention relate in general to locating sites within blood vessels. More specifically, some applications of the present invention relate to systems that facilitate mapping blood vessels and the use of electrodes at sites within the blood vessels.

### BACKGROUND

Hypertension is a prevalent condition in the general population, particularly in older individuals. Sympathetic nervous pathways, such as those involving the renal nerve, are known to play a role in regulating blood pressure. Ablation of renal nerve tissue from the renal artery is a known technique for treating hypertension.

### SUMMARY OF THE INVENTION

A system is provided for facilitating planning and/or performing nerve ablation techniques. The system comprises a control unit, an electrode catheter, and a blood pressure sensor. The control unit may be used for one or more subroutines: In a map-building subroutine an operator (e.g., a physician) builds a schematic representation of vasculature of a subject, by interacting with the control unit via a graphical user interface (GUI) displayed on a display by the control unit. In a hot-spot locating subroutine, target sites in the vasculature are located using the electrode catheter and the blood pressure sensor, and the resulting data is stored in an association with the target sites. In an ablation subroutine, the map and the stored data are used to facilitate ablation of nerve tissue at the target sites.

There is therefore provided, in accordance with an application of the present invention, a method for use with a subject, the method including:

using at least one computer processor:
generating a map of vasculature of the subject, by:
   displaying, on a display, (i) a schematic representation of a vasculature of the subject that includes a blood vessel of the subject, and (ii) on the schematic representation of the blood vessel, a first plurality of branch-site options;
   receiving a user-selected first branch site from the first plurality of branch-site options, and responsively thereto displaying, on the display, (i) a schematic representation of a first branch of the vasculature that branches, at the user-selected first branch site, at an angle from the schematic representation of the blood vessel, and (ii) a second plurality of branch-site options; and
   receiving a user-selected second branch site from the second plurality of branch-site options;
at least partially in response to receiving the user-selected second branch site, displaying the map;
detecting, using a sensor, a physiological parameter of the subject; and
in response to the detected physiological parameter:
   determining a value for the parameter, and
   storing, in association with a location on the map,
data representative of the value.

In an application, the displayed blood vessel, first branch, and second branch collectively define at least part of a vasculature map, and the method further includes storing the vasculature map on a non-transitory computer-readable medium.

In an application, the method further includes:
displaying an angle-selection input element; and
receiving a user-selected angle via the angle-selection input element,
   and displaying the schematic representation of the first branch includes configuring the displayed angle in response to the user-selected angle.

In an application, the method further includes:

displaying an angle-selection input element; and

receiving a user-selected angle via the angle-selection input element,

and displaying the schematic representation of the first branch includes displaying the schematic representation of the first branch extending at the user-selected angle from the schematic representation of the blood vessel.

In an application, the method further includes:

displaying a branch-selection input element including a plurality of schematic representations of branch types; and

receiving a user-selected branch type from the plurality of schematic representations of branch types,

and displaying the schematic representation of the first branch includes displaying the user-selected branch type.

In an application, the method further includes:

displaying a length-selection input element; and

receiving a user-selected length via the length-selection input element, and displaying the schematic representation of the first branch includes displaying a length of the schematic illustration of the first branch in response to the user-selected length.

In an application, the method further includes receiving a user-inputted location on the selected branch, and storing the data in association with the location includes automatically storing the data in association with the location in response to receiving the user-inputted location.

In an application, the method further includes, using the at least one computer processor, driving an electrode of an electrode catheter to apply an application of excitatory current, and determining the value for the parameter includes determining a value of a response of the parameter to the application of excitatory current.

In an application, the method further includes, prior to storing the data in association with the location, holding the data without storing the data in association with the location until the user-inputted location is received, and storing the data in association with the location includes storing the held data in association with the location in response to receiving the user-inputted location.

In an application, the method further includes, using the at least one computer processor, in response to the value of the detected physiological parameter, defining the location as a target site for subsequent application of ablation energy.

In an application, the method further includes, using the at least one computer processor, in response to the value of the detected physiological parameter, determining at least one ablation energy parameter for subsequent application of ablation energy.

In an application, the data representative of the value includes the determined ablation energy parameter, and storing, in association with the user-inputted location, the data representative of the value, includes storing, in association with the user-inputted location, the data that includes the determined ablation energy parameter.

There is further provided, in accordance with an application of the present invention, apparatus, for use with a vasculature of a subject, the apparatus including:

an intravascular tool, having a distal portion that includes an electrode and is configured to be transluminally advanced into the vasculature;

a blood pressure sensor;

a display; and

a control unit:
including at least one computer processor, and
interfaced with the tool, the blood pressure sensor, and the display, and
configured to:
   drive the electrode to apply an application of excitatory current, such that if the electrode is within the vasculature, the electrode applies the excitatory current to the vasculature;
   automatically detect, using the sensor, a change in blood pressure of the subject induced by the application of the excitatory current;
   display, on the display, a schematic representation of the vasculature;
   receive a user-inputted location on the schematic representation of the vasculature; and
   in response to (i) the user-inputted location, and (ii) the detected change in the physiological parameter, store, in association with the user-inputted location, data representative of the change in blood pressure.

In an application, the control unit is configured to configure the excitatory current to be an excitatory current having a frequency of 1-300 Hz, and configured to induce action potentials in nerve tissue of the vasculature.

In an application, the control unit is configured:
to (i) determine a degree of electrical contact between the electrode and the vasculature of the subject, and (ii) use the sensor to determine a blood pressure stability of the subject; and

in response to determining that (i) the degree of electrical contact is above a threshold degree of electrical contact, and (ii) the blood pressure stability is above a threshold degree of blood pressure stability, to drive the electrode to apply the application of excitatory current.

In an application, the control unit is configured:

to (i) determine a degree of electrical contact between the electrode and the vasculature of the subject, and (ii) use the sensor to determine a blood pressure stability of the subject;

in response to determining that (i) the degree of electrical contact is above a threshold degree of electrical contact, and (ii) the blood pressure stability is above a threshold degree of blood pressure stability, to enable a user-operated switch; and in response to operation of the switch, to drive the electrode to apply the application of excitatory current.

There is further provided, in accordance with an application of the present invention, a method for use with a subject, the method including, using at least one computer processor:

displaying, on a display, a schematic representation of a vasculature of the subject;

driving an electrode of an electrode catheter, disposed within the vasculature, to apply an application of excitatory current to the vasculature;

detecting, using a sensor, a change in a physiological parameter of the subject induced by the application of excitatory current;

receiving a user-inputted location on the schematic representation; and

in response to (i) the user-inputted location, and (ii) the detected change in the physiological parameter, storing, in association with the user-inputted location, data representative of the change in the physiological parameter.

In an application, the method further includes, displaying, on the display, an indication of the association of the data representative of the physiological parameter with the location.

In an application, storing the data in association with the user-inputted location includes defining the user-inputted location as a target site for subsequent application of ablation energy.

In an application, the user-inputted location is a first user-inputted location, the application of excitatory current is a first application of excitatory current, the change is a first change, and the method further includes, using the at least one computer processor:

driving the electrode of the electrode catheter, disposed within the vasculature, to apply a second application of excitatory current to the blood vessel;

detecting, using the sensor, a second change in a physiological parameter of the subject induced by the second application of excitatory current;

receiving a second user-inputted location of the at least one blood vessel; and

in response to (i) the second user-inputted location, and (ii) the second detected change in the physiological parameter, storing, in association with the second user-inputted location, data representative of the second change in the physiological parameter.

In an application, the method further includes, using the processor, detecting a degree of electrical contact between the electrode and the vasculature, and displaying, on the display, a contact-quality indicator that indicates the detected degree of electrical contact, and driving the electrode includes driving the electrode in response to receiving a user-inputted initiation that is inputted while the degree of electrical contact is above a threshold degree of electrical contact.

In an application, the method further includes using the processor to determine a stability of blood pressure of the subject by using the sensor, and displaying, on the display, a blood-pressure-stability indicator that indicates the determined stability, and driving the electrode includes driving the electrode in response to receiving a user-inputted initiation that is inputted while stability is greater than a threshold stability.

In an application, the user-inputted location is a first user-inputted location, the application of excitatory current is a first application of excitatory current, the change is a first change, and the method further includes, using the at least one computer processor:

driving the electrode of the electrode catheter, disposed within the vasculature, to apply a second application of excitatory current to the blood vessel;

detecting, using the sensor, a second change in a physiological parameter of the subject induced by the second application of excitatory current; and

receiving a second user-inputted location of the at least one blood vessel,

and storing the data in association with the user-inputted location includes location includes, in response to (i) the first user-inputted location, (ii) the first detected change in the physiological parameter, (iii) the second user-inputted location, and (iv) the second detected change in the physiological parameter, defining, as a target site for subsequent application of ablation energy, at least one user-inputted location selected from the group consisting of: the first user-inputted location and the second user-inputted location.

In an application, the method further includes, using the processor, comparing the first detected change and the second detected change, and defining the selected user-inputted location as the target site includes defining the user-inputted location as the target site in response to the comparing.

There is further provided, in accordance with an application of the present invention, a method for use with a subject, the method including:

advancing an electrode to a site in a vasculature of the subject;

advancing an intravascular blood pressure sensor into the subject;

activating a control unit to (i) drive the electrode to apply an excitatory current to the site, and (ii) detect a change in blood pressure of the subject induced by the excitatory current;

subsequently, viewing a schematic representation of the vasculature displayed by the control unit; and

subsequently, by selecting a location on the schematic representation of the vasculature, activating the control unit to store, in association with the location, data representative of the change.

There is further provided, in accordance with an application of the present invention, a method for use with a subject, the method including, using at least one computer processor:

detecting a first degree of electrical contact between a plurality of electrodes and tissue of the subject;

only if the first degree of electrical contact is above a contact threshold, initiating driving the electrodes to apply excitatory current to the tissue;

after the start of the application of excitatory current, detecting (i) a second degree of electrical contact between the electrodes and the tissue, and (ii) a blood pressure change since the initiating of the driving of the electrodes;

determining whether (i) the second degree of electrical contact is below the contact threshold and (ii) the detected blood pressure change is greater than a pressure-change threshold; and

in response to determining that (i) the second degree of electrical contact is below the contact threshold and (ii) the detected blood pressure change is greater than the pressure-change threshold, continuing to drive at least one of the electrodes to apply excitatory current.

There is further provided, in accordance with an application of the present invention, a method for use with a subject, the method including:

viewing, on a display of a system, (i) a schematic representation of vasculature of the subject, and (ii) a plurality of indicators of respective target sites within the vasculature;

by selecting one of the target sites using the system, activating the system to retrieve ablation energy parameters specific for the one of the target sites;
transluminally advancing a tool that includes an electrode at a distal portion thereof, into the vasculature of the subject, such that the electrode becomes disposed at the selected site; and

activating the system to drive the electrode to apply ablation energy using the parameters specific for the selected site.

### BRIEF DESCRIPTION OF THE DRAWINGS

FIG. 1 is a schematic illustration of a system being used with a subject, in accordance with some applications of the invention;

FIG. 2 is a flowchart that shows at least some steps of a workflow performed using the system;

FIGS. 3A-J are schematic illustrations showing a graphical user interface (GUI) displayed on a display of the system during a map-building subroutine, in accordance with some applications of the invention;

FIGS. 4A-G are schematic illustrations showing an alternative GUI displayed on the display during the map-building subroutine, in accordance with some applications of the invention;

FIG. 5 is a flowchart showing at least some steps of the map-building subroutine, in accordance with some applications of the invention;

FIG. 6 is a flowchart showing at least some steps of a hotspot-locating subroutine, in accordance with some applications of the invention;

FIGS. 7A-K are schematic illustrations showing a graphical user interface (GUI) displayed on the display during the hotspot-locating subroutine, in accordance with some applications of the invention;

FIG. 8 is a flowchart showing at least some steps of an ablation subroutine, in accordance with some applications of the invention;

FIGS. 9-11 are flowcharts showing at least some steps of respective algorithms performed by a control unit of the system, in accordance with an application of the inventions; and

FIG. 12 is a lookup table for stimulation decisions, in accordance with some applications of the invention.

### DETAILED DESCRIPTION OF EMBODIMENTS

Reference is made to FIG. 1, which is a schematic illustration of a system 20 being used with a subject 5, in accordance with some applications of the invention. System 20 comprises an intravascular tool 30, a display 40, and a control unit 50. Tool 30 comprises a catheter 32 that has a distal portion that comprises at least one electrode 34, and is transluminally advanceable into a blood vessel of subject 5, such as the renal artery 10 of the subject. Typically, tool 30 comprises a reversibly-expandable electrode device 36 on which electrode 34 is mounted. System 20 also comprises a blood pressure sensor 38, such as an intravascular pressure sensor (as shown), e.g., disposed on catheter 32. Alternatively, pressure sensor 38 may be an extracorporeal blood pressure sensor, such as a blood pressure cuff.

Control unit 50 comprises at least one computer processor 52, a catheter interface (e.g., a port, such as a socket) 54 via which the control unit interfaces with tool 30, and a display interface (e.g., a port, such as a socket) 56 via which the control unit interfaces with display 40. Control unit 50 comprises a pressure-sensor interface (e.g., a port, such as a socket) 60 via which the control unit interfaces with pressure sensor 38. For some applications (e.g., for applications in which pressure sensor 38 is disposed on catheter 32), interface 60 and interface 54 may be integrated with each other, or may be subcomponents of a common interface. Alternatively (e.g., for applications in which pressure sensor 38 is a blood pressure cuff), interfaces 54 and 60 may be distinct from each other (e.g., may comprise separate connections for catheter 32 and sensor 38).

Control unit 50 typically also comprises at least one memory 58, which may be physically located within a common housing of the control unit, or may be located elsewhere and connected to processor 52 e.g., via a network. For some applications, memory 58 comprises one or more of the following: a semiconductor or solid state memory, magnetic tape, a removable computer diskette, a random access memory (RAM), a read-only memory (ROM), a rigid magnetic disk, and an optical disk.

Control unit 50 comprises at least one user input device 42, such as a mouse, keyboard, or trackball. For some applications, user input device 42 is integrated into display 40 as a touchscreen. It is to be understood that the scope of the invention includes the use of any appropriate input device known in the art. The operation of control unit 50 by the operator, described hereinbelow, are implemented via user input device 42.

System 20 facilitates the planning (and optionally the performing) of transluminal nerve ablation procedures; typically renal nerve ablation procedures, which are also known as renal denervation (RDN) procedures. System 20 provides the operating physician(s) with a schematic representation (e.g., a map, described hereinbelow) of the vasculature of the subject, for use during planning (and optionally performing) RDN procedures. The term “schematic” in this context (including the specification and the claims) means symbolic and simplified. It is hypothesized by the inventors that, compared with a more true-to-life representation such as an x-ray or ultrasound image, such a schematic representation provides a simpler guide for the operating physician(s) to follow, resulting in more consistent interpretation of the representation (and therefore navigation through the vasculature) by different physicians.

Functions (e.g., routines and subroutines) of system 20 (e.g., control unit 50 thereof) are described hereinbelow.

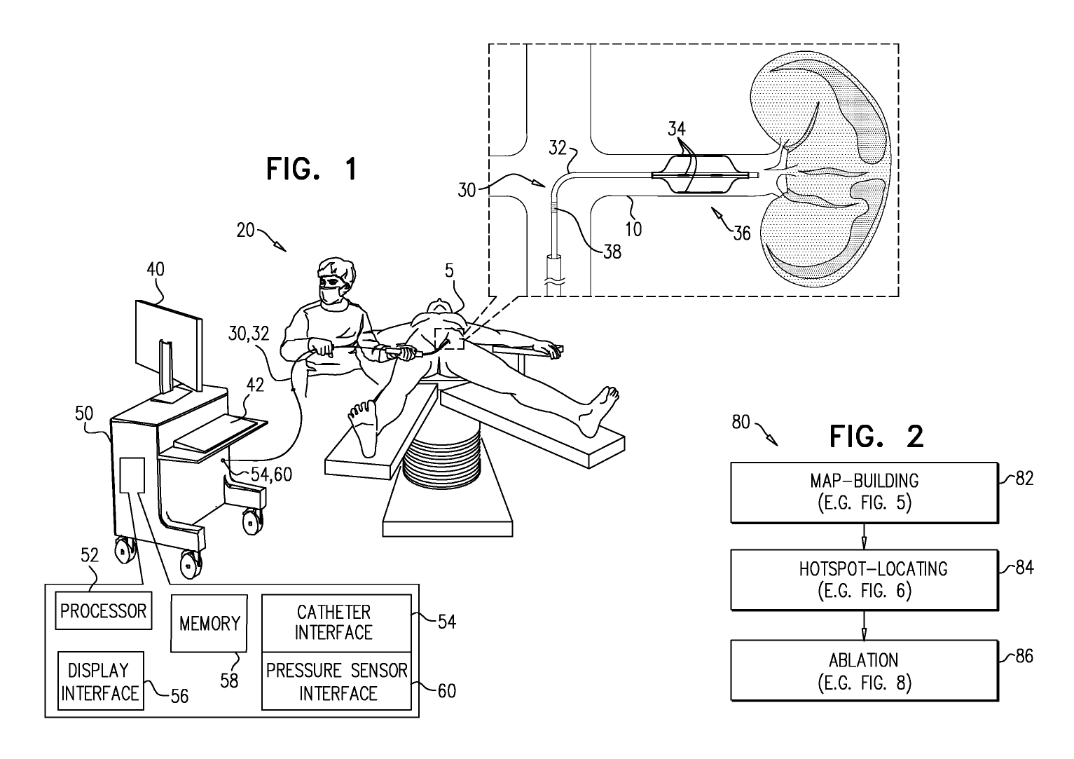

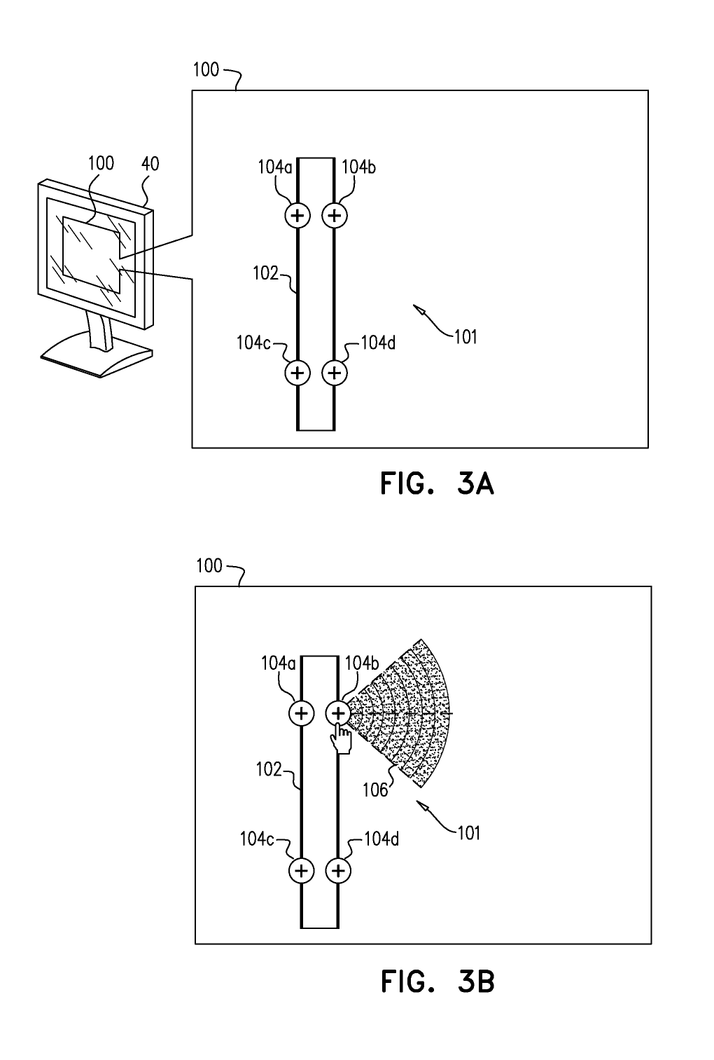

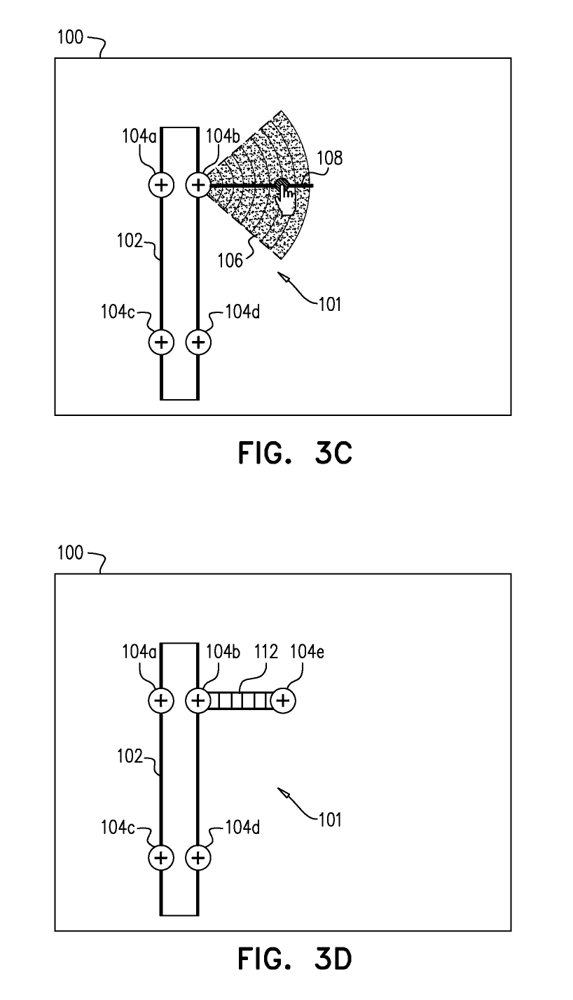

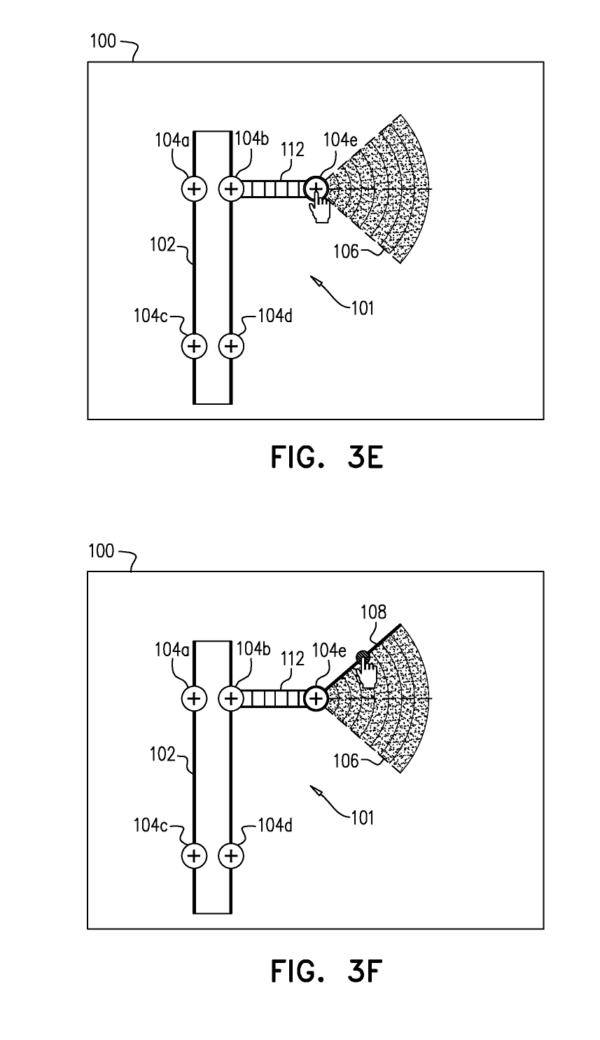

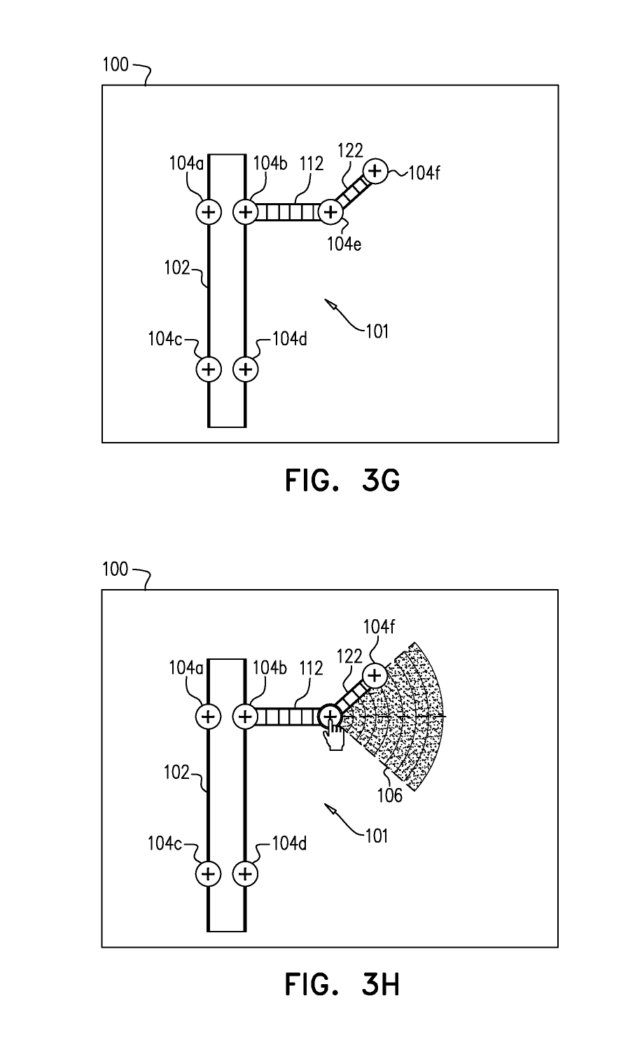

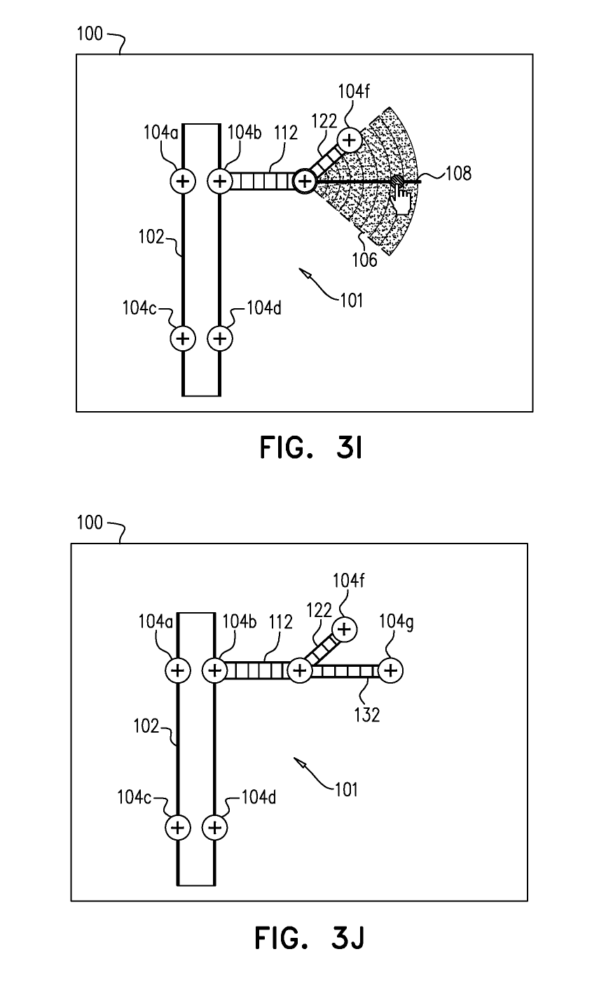

Reference is made to FIG. 2, which is a flowchart that shows at least some steps of a workflow 80 performed using system 20. For some applications, a map-building subroutine (e.g., an algorithm) 82 (described hereinbelow with respect to FIGS. 3A-J and 4A-G) is used, in order to build a schematic map of vasculature (e.g., the renal vasculature) of the subject.

The operator (e.g., a physician) typically builds the map during subroutine 82, facilitated by an image of the vasculature that is to be mapped. For example, the physician may refer to an image generated using fluoroscopy, MRI, or another imaging technique. The image may be on paper or film, or displayed on a display. For some applications, the image is displayed on display 40, and is overlaid with graphical elements and representations used for the map building (e.g., those described with reference to FIGS. 3A-J and 4A-G).

For some applications, subroutine 82 is not used, e.g., because the schematic map is already available (e.g., stored in a memory 58). For example, the schematic map may have been built previously using subroutine 82 (e.g., by the operating physician, by another operator, or even at a different facility). Alternatively, the schematic map may have been generated automatically (e.g., by processing of an image of the anatomy—e.g., obtained by x-ray, ultrasound, etc.).

Subroutine (e.g., an algorithm) 84 tests test sites of artery 10 in order to identify target sites that are suitable for nerve ablation, and for some applications also determines ablation parameters (e.g., ablation power, duration and modality) for each of the target sites. Testing is performed by applying excitatory current to each test site, and detecting a response thereto (e.g., a change in blood pressure, such as Mean Arterial Pressure (MAP)). A response of sufficient magnitude indicates that the test site is sufficiently close to a nerve fiber for ablation energy applied at the site to ablate the nerve fiber, and therefore that the test site is a target site suitable for nerve ablation. Control unit 50 (e.g., processor 52 thereof) stores this location of the target sites (and optionally the ablation parameters) in association with the schematic map, for use during an ablation subroutine (e.g., an algorithm) 86. Ablation subroutine 86 may be performed by the operating physician (e.g., soon after subroutine 84), by another operator, or even at a different facility.

The schematic map, and the data stored in association with it (e.g., the location on the map of the target sites, and ablation parameters) are stored in memory 58.

Reference is made to FIGS. 3A-J, which are schematic illustrations showing a graphical user interface (GUI) 100 displayed on display 40 during map-building subroutine 82, in accordance with some applications of the invention. Display 40 displays, inter alia, a schematic map 101 of the vasculature of the subject. Display 40 typically also displays other GUI items, such as control items, including navigation items that facilitate transitioning between subroutines 82, 84 and 86.

A schematic representation of a blood vessel 102 (or at least a portion thereof) is displayed on display 40 (e.g., as part of map 101) (FIG. 3A). For example, when system 20 is used for facilitating RDN, the represented blood vessel may be the abdominal aorta of the subject. A plurality of branch-site options 104 (e.g., branch site options 104a, 104b, 104c and 104d) are displayed on representation 102.

An operator (i.e., a user, e.g., a physician) selects (i.e., inputs) one of branch site options 104 (in this case branch site 104b), and in response to receiving this selection, control unit 50 displays a schematic representation of a branch 112 that branches, at site 104b, from schematic representation 102 (FIGS. 3B-D). Typically, subroutine 82 provides for the operator to select the angle and/or length of branch 112. For example, in response to receiving this user-selected branch site, an angle-selection input element 106 is displayed, and the operator selects an angle of branch 112. Alternatively or additionally, in response to receiving the user-selected branch site, a length-selection input element 108 is displayed, and the operator selects a length of branch 112. FIGS. 3B-C show an example in which both input elements 106 and 108 are used, and in which the input elements show the angle and length juxtaposed with site 104b, such that when branch 112 is eventually displayed (FIG. 3D), the branch is displayed at the same position and angle on display 40 as the user-selected length and angle.

A second plurality of branch-site options 104 is displayed (FIG. 3D), typically including one or more branch-site options 104 on branch 112, and further typically including at least some (e.g., all) of the branch site options from the first plurality of branch site options. The operator selects a second branch site from the second plurality of branch-site options (e.g., branch site 104e, as shown), and in response to the selection, a schematic representation of a second branch 122 is displayed (FIGS. 3E-G). For some applications, angle-selection input element 106 and/or a length-selection input element 108 is displayed and used as described with reference to FIGS. 3B-D, mutatis mutandis.

FIG. 3J shows a schematic representation of a third branch 132 having been added to map 101 at branch site 104e as described for branches 112 and 122, mutatis mutandis (FIGS. 3H-I).

Collectively, the schematic representations of vessel 102 and branches 112 and 122 define at least part of a schematic vasculature map 101. Vasculature map 101 may be subsequently used for hotspot-locating subroutine 84, or may be stored in memory 58 for future use.

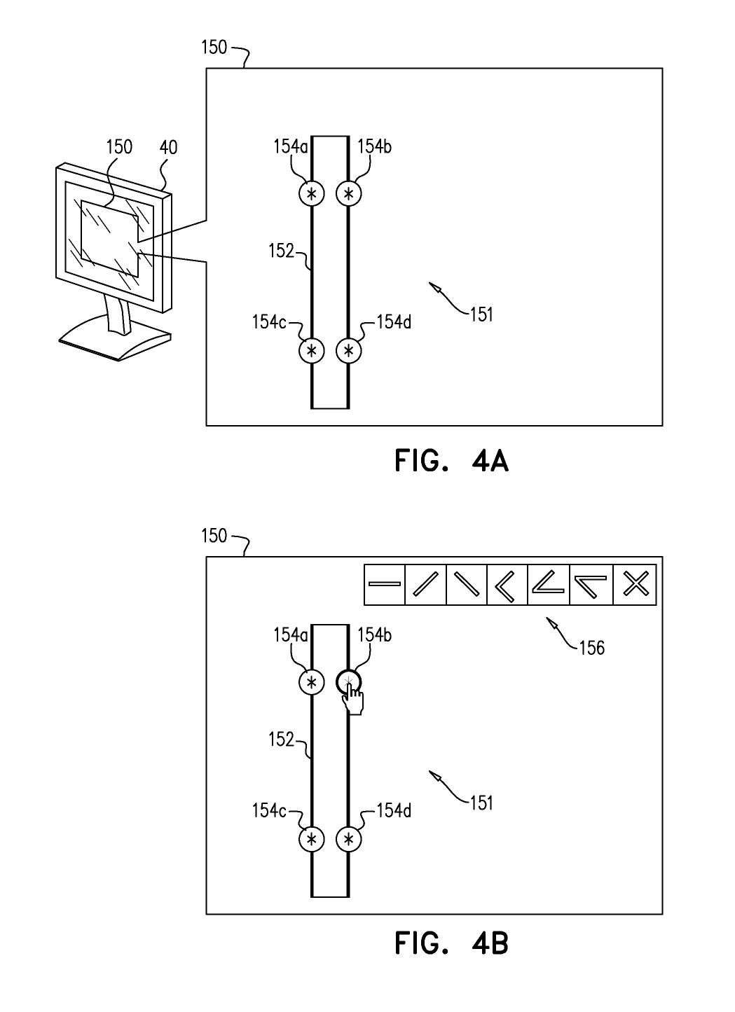

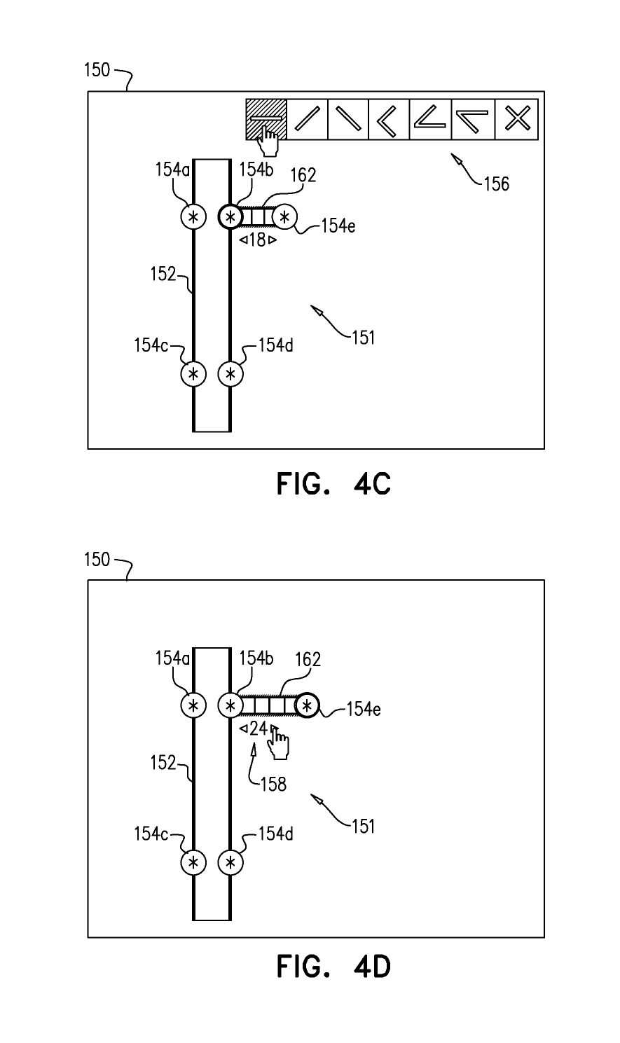

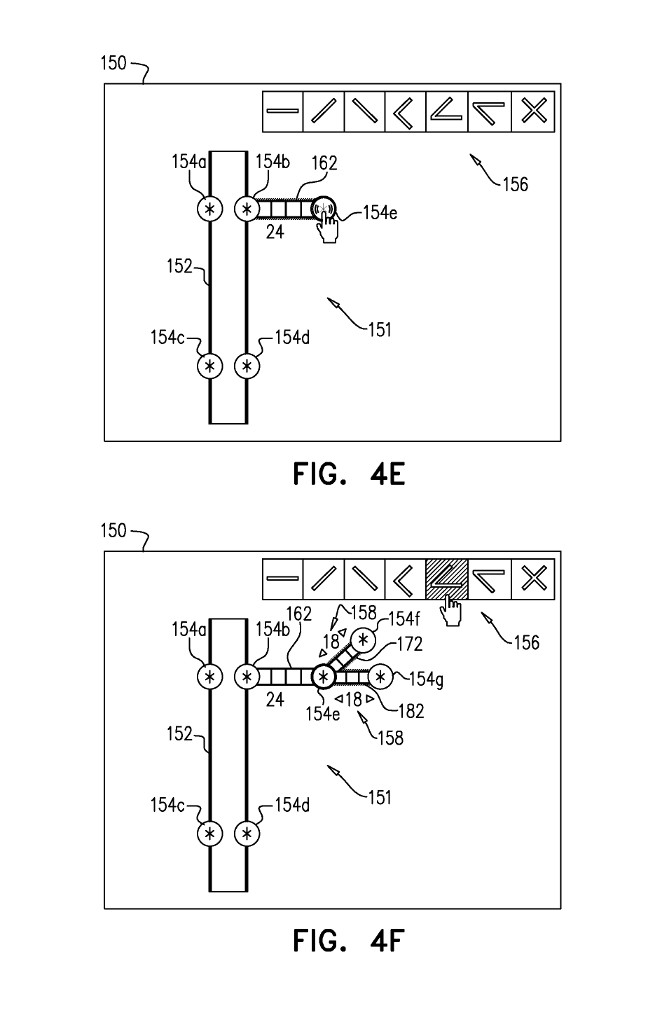

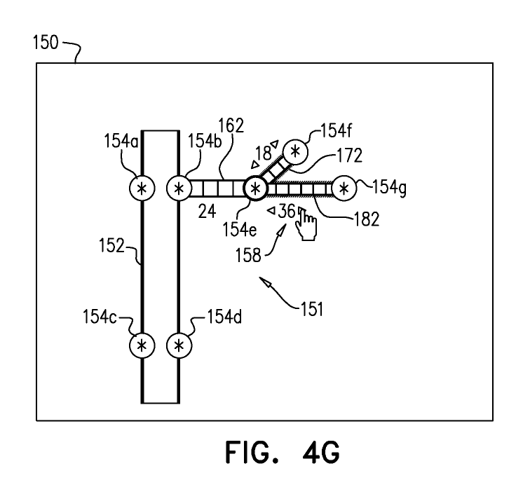

Reference is now made to FIGS. 4A-G, which are schematic illustrations showing an alternative GUI 150 displayed on display 40 during map-building subroutine 82, in accordance with some applications of the invention. GUI 150 shares similarities with GUI 100, including displaying a schematic map 151 of the vasculature of the subject, that includes a blood vessel 152, branch site options 154, an angle-selection input element 156, a length-selection input element 158, and the resulting branches 162, 172 and 182.

Angle-selection input element 156 comprises a plurality of schematically represented choices of branch types, from which the operator selects an appropriate branch type. Therefore element 156 may alternatively or additionally be referred to as a branch-selection input element. Length-selection input element 158 indicates the length of the relevant branch, and provides buttons for increasing and decreasing this length. The different angle-selection input elements and length-selection input elements described with reference to FIGS. 3A-J and 4A-G are intended as illustrative examples of such elements, but it is to be understood that the scope of the invention includes the use of other types of interface elements.

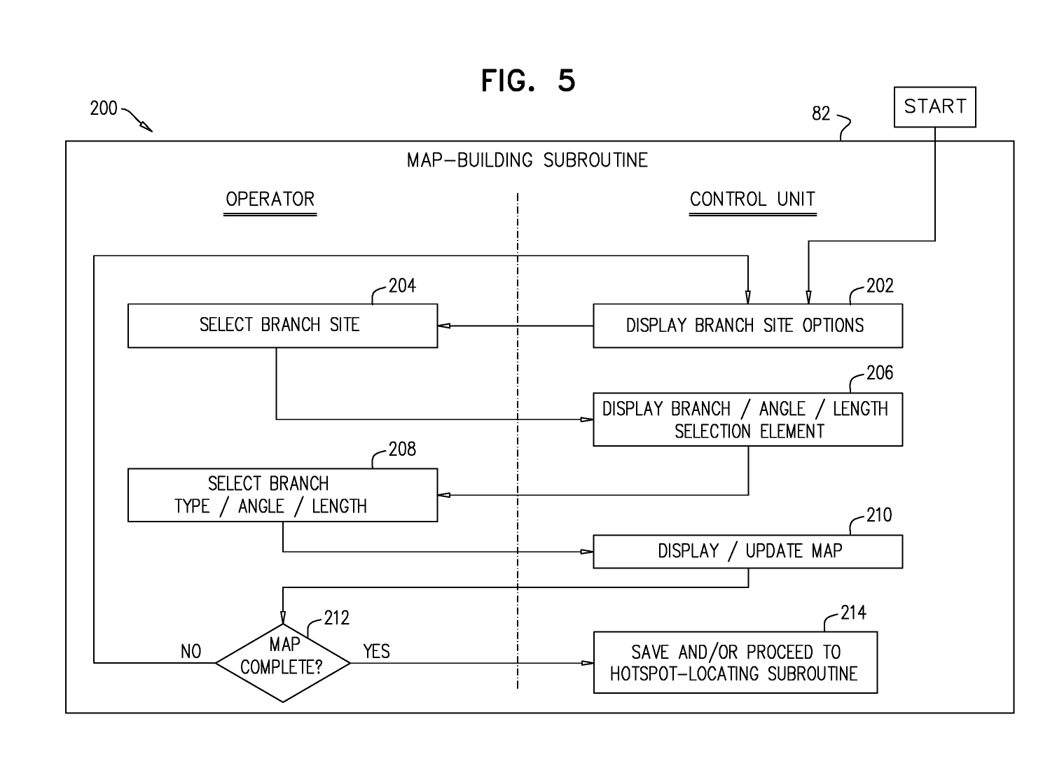

Reference is further made to FIG. 5, which is a flowchart showing at least some steps of subroutine 82 (e.g., the techniques described with reference to FIGS. 3A-J and 4A-G), in accordance with some applications of the invention. The flowchart is arranged to distinguish between steps performed by the operator, e.g., the physician (on the left side of the flowchart), and those performed by control unit 50 (on the right side of the flowchart).

In step 202, control unit 50 displays, on display 40, a plurality of branch site options (as well as the schematic representation of the blood vessel). For example, step 202 may relate to FIGS. 3A and 4A and the descriptions thereof. In step 204, the operator selects a branch site, e.g., as described with reference to FIGS. 3B and 4B, mutatis mutandis. In step 206, (typically in response to the selection of the branch site, although optionally independently of the selection of the branch site), control unit 50 displays a branch-selection element, angle-selection element, and/or length-selection element, and in step 208 the operator selects a branch type, branch angle, and/or branch length using the corresponding selection element. Steps 206 and 208 relate to FIGS. 3B-C and 4B-4D, mutatis mutandis. Responsively, control unit 50 displays the map, updated to include the schematic representation of the newly-added branch—in this case, branch 112 (step 210).

Steps 202-208 are repeated until the schematic map of the vasculature is complete (represented by decision 212), at which point the map is typically saved to memory 58, and optionally the operator and system 20 proceed to hotspot-locating subroutine 84 (step 214).

Therefore, with reference to FIGS. 3A-J, 4A-G, and 5, a method is described of using at least one computer processor to:
(a) display, on a display, (i) a schematic representation of a vasculature of a subject that includes a blood vessel of the subject, and (ii) on the schematic representation of the blood vessel, a first plurality of branch-site options;
(b) receive a user-selected first branch site from the first plurality of branch-site options, and responsively thereto display, on the display, (i) a schematic representation of a first branch of the vasculature that branches, at the user-selected first branch site, at an angle from the schematic representation of the blood vessel, and (ii) a second plurality of branch-site options; and
(c) receive a user-selected second branch site from the second plurality of branch-site options, and responsively display a schematic representation of a second branch of the vasculature branching at the user-selected second branch site.

Furthermore, the method typically comprises using the computer processor to display a length-selection input element, an angle-selection input element, and/or a branch-selection input element, and responsively displaying or configuring one of the schematic representations of the branches accordingly.

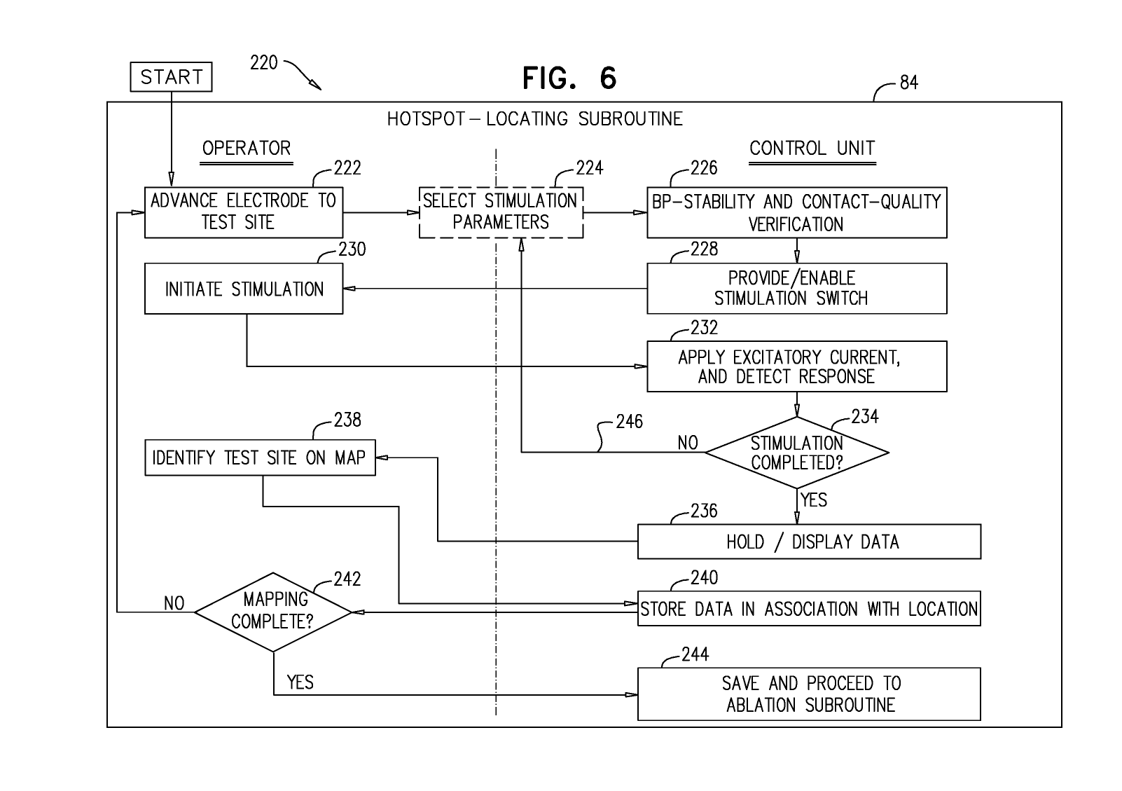

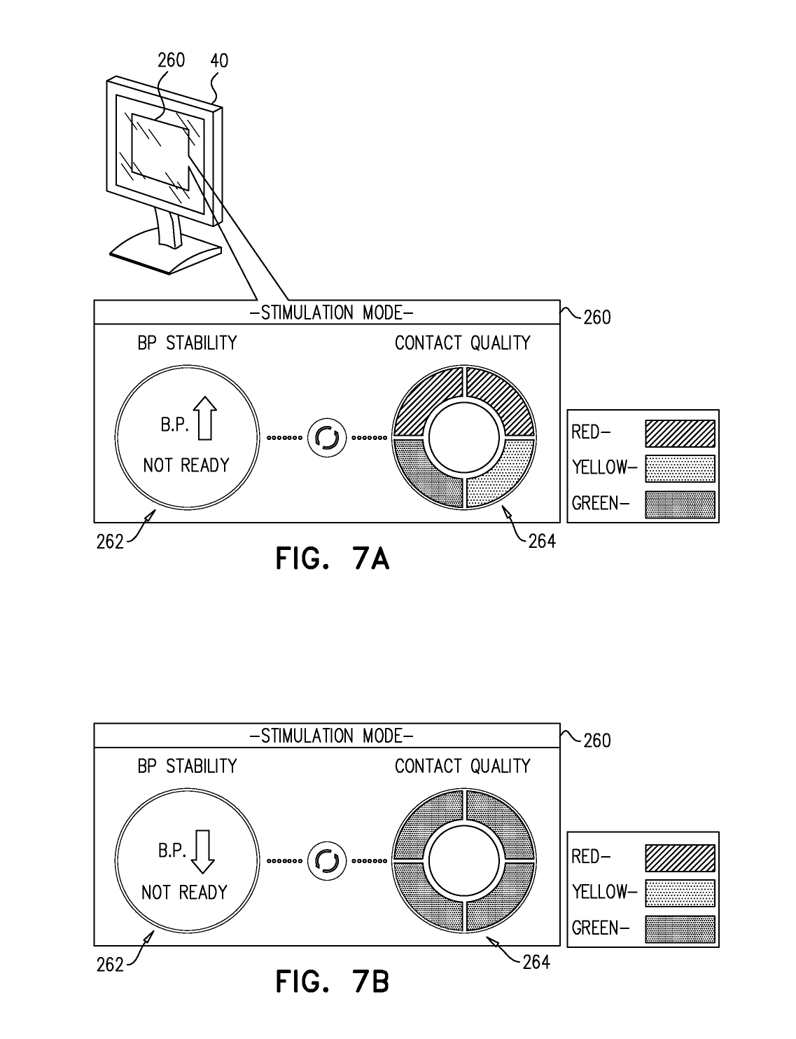

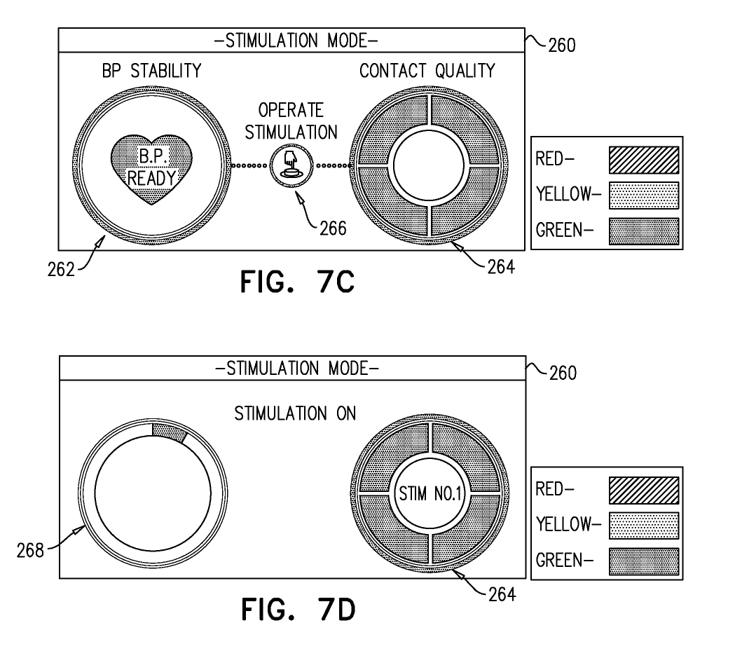

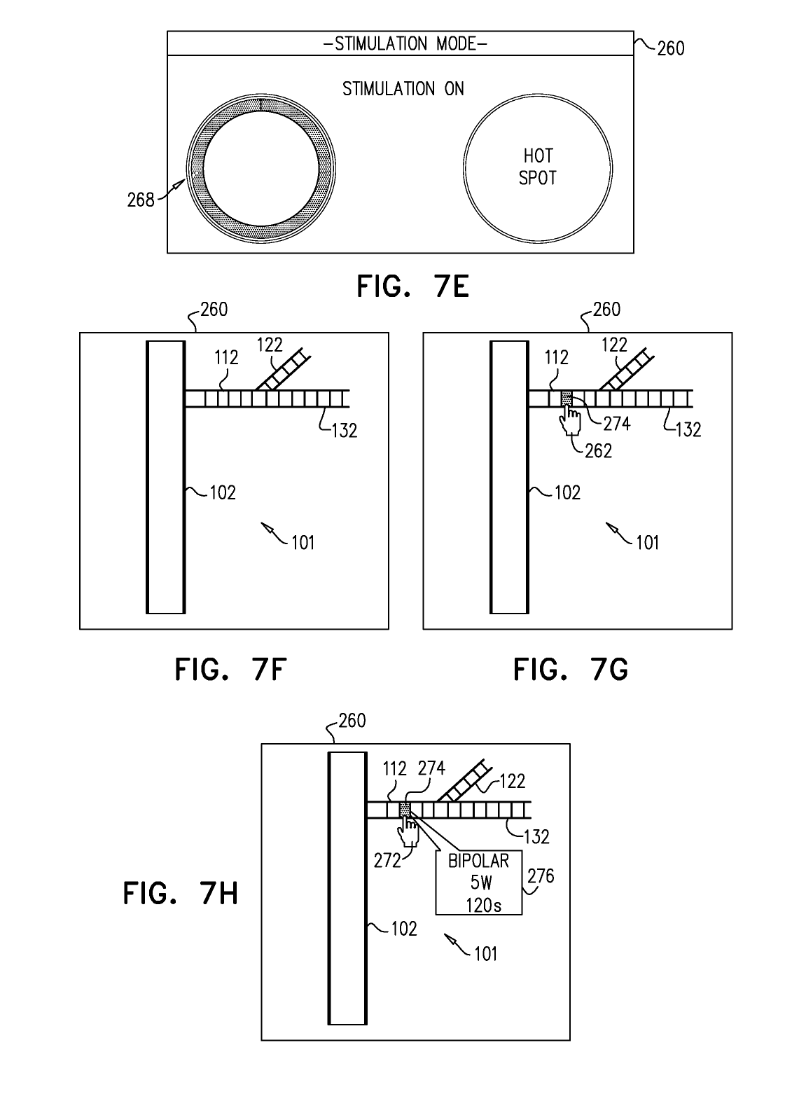

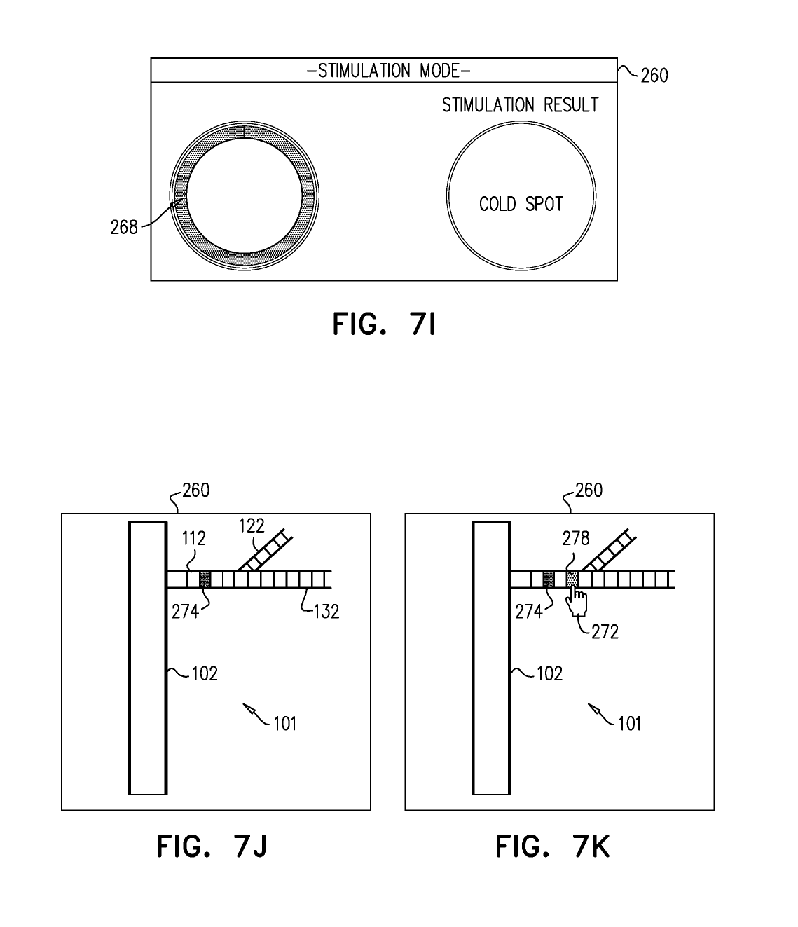

Reference is made to FIG. 6, which is a flowchart 220 showing at least some steps of subroutine 84, in accordance with some applications of the invention. The flowchart is arranged to distinguish between steps performed by the operator, e.g., the physician (on the left side of the flowchart), and those performed by control unit 50 (on the right side of the flowchart). Reference is also made to FIGS. 7A-K, which are schematic illustrations showing a graphical user interface (GUI) 260 displayed on display 40 during hotspot-locating subroutine 84, in accordance with some applications of the invention. Display 40 typically also displays other GUI items, such as control items.

The operator advances intravascular tool 30 transluminally (e.g., transfemorally), such that electrode 34 is disposed at a test site that is to be tested in order to determine if the test site is a suitable target site for nerve ablation (step 222). Optionally, stimulation parameters are selected, e.g., by the operator, or automatically by control unit 50 (step 224). Step 224 is described in more detail hereinbelow.

Typically, control unit 50 performs verification of blood pressure (BP) stability and/or quality of electrical contact between electrode 34 and the blood vessel wall (step 226). Verification of

BP stability is performed using the blood pressure detected by pressure sensor 38, and received via pressure-sensor interface 60. It is hypothesized by the inventors that verifying BP stability (i.e., that there is little or no change in BP) prior to testing the test site facilitates identification and measurement of a response to the subsequently-applied excitatory current, by eliminating background changes in BP from the detected response. Typically, and as described hereinbelow, control unit 50 performs the electrical contact quality verification continuously throughout steps 228, 230 and 232.

FIGS. 7A-B show GUI 260 during step 226. A blood-pressure-stability indicator 262 indicates blood pressure stability, and a contact-quality indicator 264 indicates a quality (i.e., a degree) of electrical contact between electrode 34 and the blood vessel wall. For some applications, electrode device 36 comprises a plurality of electrodes 34 (e.g., four electrodes 34, as shown), and the contact quality of each electrode is indicated independently.

Two techniques for verifying contact quality of electrodes 34 are briefly described:

Technique (1): In some applications of the present invention, control unit 50 (e.g., processor 52 thereof) is configured to apply electrical pulses between a pair of electrodes, including at least one intrarenal electrode (e.g., an electrode 34); calculate at least one time-varying component of electrode-tissue impedance based on applying the pulses; sense a periodic hemodynamic signal of the subject; calculate a level of correlation between the at least one time-varying component of the electrode-tissue impedance and the periodic hemodynamic signal; and, based on the level of correlation, ascertain a level of electrical contact between the at least one intrarenal electrode and the wall of the renal artery. Typically, and as described hereinabove, control unit 50 displays the level of contact on display 40. The periodic hemodynamic signal and the at least one time-varying component of the electrode-tissue impedance may correlate because of local mechanical changes in the blood vessel wall caused by periodic variations in blood pressure.

For some applications, the periodic hemodynamic signal is blood pressure of the subject (e.g., intravascular blood pressure, or an external measurement of blood pressure). For some applications, control unit 50 (e.g., processor 52 thereof) is configured to calculate the level of correlation between the at least one time-varying component of the electrode-tissue impedance and the periodic hemodynamic signal by analyzing a phase difference between the at least one time-varying component of the electrode-tissue impedance and the periodic hemodynamic signal.

For some applications, the at least one time-varying component of the electrode-tissue impedance is selected from one of the following:
an electrode-tissue interface series resistance,
an electrode-tissue interface capacitance, or
a relationship (e.g., a ratio) between the electrode-tissue impedance and the electrode-tissue interface capacitance, e.g., the quotient of (a) the electrode-tissue interface series resistance divided by (b) the electrode-tissue interface capacitance (in general, when better contact is achieved, the electrode-tissue interface series resistance increases and the electrode-tissue interface capacitance decreases; the ratio thus increases with better contact).

Technique (2): In some applications of the present invention, two or more sensing electrodes are disposed in or on the body of the subject, including disposing at least one of the sensing electrodes on an external surface of skin of the subject, the sensing electrodes being separate and distinct from intrarenal current-application electrodes 34. Control unit 50 (e.g., processor 52 thereof) is configured to apply an electrical current between a pair of current-application electrodes, including at least one intrarenal current-application electrode 34. While applying the current, control unit 50 senses an electrical signal between two or more sensing electrodes, including the at least one external electrode; and, based on a property of the electrical signal, ascertains a level of contact between the at least one intrarenal current-application electrode 34 and the wall of the renal artery. Typically, and as described hereinabove, control unit 50 displays the level of contact on display 40.

For such applications, control unit 50 typically has additional electrode interfaces (e.g., ports) via which the sensing electrodes are connected to processor 52.

For some applications, the at least one external electrode comprises at least two external electrodes. For some applications, the external electrodes are conventional electrocardiogram (ECG) electrodes, which may be positioned at one or more of the conventional ECG electrode locations on the body, and which may also be used to sense an ECG of the subject.

Application of the electrical current may “confuse” an ECG monitor, which senses the applied signals and interprets the applied signals as drastic increases in heart rates. Effective application of the current (whether ablation or stimulation) results in stable interference with the ECG. In some applications of the present invention, such stable interference is interpreted as an indication of good contact between the at least one intrarenal electrode and tissue of the wall of the renal artery. For some such applications, control unit 50 serves as an ECG monitor, and is connected to the ECG electrodes via one or more electrode interfaces (e.g., ports).

For some applications, control unit 50 (e.g., processor 52 thereof) is configured to ascertain the level of contact based on a shape of a time-varying signal rate while a current is applied. For some applications, control unit 50 is configured to ascertain the level of contact based on a stability of the time-varying signal rate. For some applications, control unit 50 is configured to extract at least one plateau from a graph of the time-varying signal rate, ascertain the shape of plateau, and ascertain the level of contact based on the shape of plateau. For some applications, control unit 50 is configured to calculate a flatness of the plateau, and ascertain the level of contact based on the flatness of the plateau.

These techniques for verifying quality of electrical contact are described in more detail in U.S. patent application Ser. No. 14/794,737 to Gross et al., filed Jul. 8, 2015, which is incorporated herein by reference.

FIG. 7A indicates that blood pressure is unstable, in that it is increasing at more than a threshold rate, and FIG. 7B indicates that blood pressure is unstable, in that it is decreasing at more than a threshold rate. In the figures, indicator 262 uses arrows to indicate blood pressure stability, but it is to be understood that the scope of the invention includes other ways of indicating blood pressure stability.

For some applications, control unit 50 (e.g., processor 52 thereof) deems blood pressure to be stable if the measured blood pressure has fluctuated less than a threshold amount (e.g., less than 2 mm Hg, such as less than 1 mm Hg) during a threshold stability duration (e.g., during the previous 10 seconds, 20 seconds, or 30 seconds). Typically, the threshold stability duration is less than 60 seconds (e.g., 10-45 seconds). For some applications, control unit 50 (e.g., processor 52 thereof) runs an algorithm in which the threshold stability duration required is inversely related to measured blood pressure change. For example, control unit 50 may shorten the threshold stability duration in response to determining that the BP fluctuation is highly stable. Therefore, for some applications, control unit 50 utilizes at least two threshold stability durations.

FIG. 7A indicates that two of electrodes 34 are in poor electrical contact with the blood vessel wall (by representing these electrodes in red), one of the electrodes is in moderate electrical contact (represented in yellow), and one is in good electrical contact (represented in green). FIG. 7B indicates that all four of electrodes 34 are in good electrical contact with the blood vessel wall (represented in green), e.g., following repositioning of the tool in order to improve electrical contact.

Optionally, system 20 facilitates the operator disabling one or more of electrodes 34. For example, for applications in which the operator wishes to identify the circumferential position of nerve fibers, electrodes may be sequentially disabled and re-enabled. Disabled electrodes may, for example, be represented in white.

In the figures, indicator 264 indicates electrical contact quality of electrodes 34 using colors, but it is to be understood that the scope of the invention includes other ways of indicating electrical contact quality.

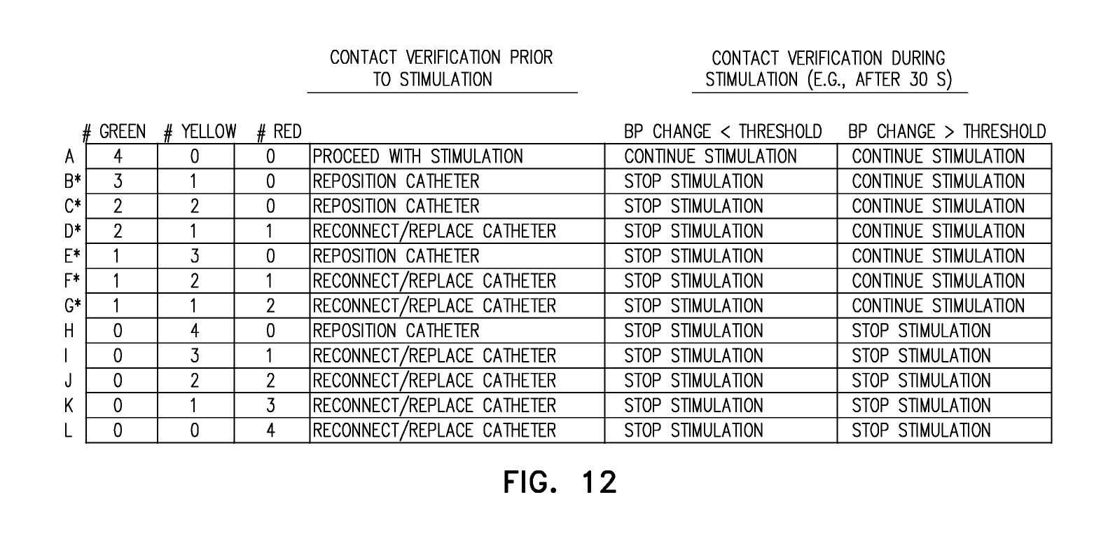

Once both BP stability and electrical contact quality are verified as sufficient, control unit 50 provides (or enables a previously disabled) switch 266 (step 228 of flowchart 220). In the example shown (and as further described hereinbelow with reference to the decision lookup table in FIG. 12), the electrode contact quality of FIG. 7A is insufficient for control unit 50 to proceed to step 228, and although the electrode contact quality of FIG. 7B is sufficient, the blood pressure stability is not. FIG. 7C shows switch 266 having been provided (e.g., displayed on display 40) by control unit 50 in response to both blood pressure stability and electrical contact quality having become sufficient. The operator is then able to initiate stimulation by operating switch 266 (step 230). In response, control unit 50 drives electrodes 34 to apply excitatory current, and detects the response (e.g., a value of the response, such as a change in blood pressure) to the excitatory current using pressure sensor 38 (e.g., via interface 60) (step 232). For some applications, and as shown in FIG. 7D, a progress indicator 268 indicates the progress of the stimulation. For some applications, a response indicator (not shown) indicates the ongoing/real-time response to the excitatory current (e.g., the change in BP).

Although FIG. 6 shows otherwise, for some applications, step 230 is performed automatically by control unit 50 in response to the same BP stability and contact quality conditions (e.g., immediately upon these conditions being met). For some such applications, step 228 is omitted.

It is to be noted that switch 266 may be a displayed switch (as shown in FIG. 7C), but may alternatively be a distinct hardware switch.

During the stimulation, control unit 50 typically continues to verify electrical contact quality. For some applications, and as also shown in FIG. 7D, contact-quality indicator 264 is displayed during the stimulation. As represented by decision 234, should electrical contact quality drop below a threshold quality during the application of excitatory current (i.e., before the application of excitatory current is completed), control unit 50 returns to step 226 (optionally including step 224 prior to step 226). Otherwise, (i.e., if the application of excitatory current is successfully completed), control unit progresses to step 236.

Reference is now further made to FIG. 12, which is a lookup table for stimulation decisions, in accordance with some applications of the invention. For some applications, in decision 234, if electrical contact quality of a particular electrode drops below a threshold quality during the application of excitatory current, control unit 50 nonetheless continues to apply the excitatory current. Control unit 50 may do this even in a case in which the overall electrical contact quality of all the electrodes is lower than would have been required to initiate the application of excitatory current (e.g., lower than would have been required for control unit 50 to provide and/or enable the stimulation switch).

Each row of FIG. 12 represents a scenario in which electrical contact quality has been tested. The first three columns of the table represent the result of the test, indicating the number of electrodes (in this case from a total of 4 electrodes) that have been determined to have good (green), moderate (yellow), and poor (red) electrical contact with the tissue. Collectively, these results for each row are referred to herein as contact quality scenarios. The fourth column indicates the decision to be made if the test that identified the particular contact quality scenario was performed prior to stimulation (e.g., during step 226 of subroutine 84).

The fifth and sixth columns indicate the decision to be made if the test that identified the contact quality scenario was performed during stimulation (i.e., before stimulation is completed), such as after 30 s of stimulation (e.g., during decision 234 of subroutine 84). The fifth column indicates the decision to be made if, at that time, the blood pressure of the subject has not increased more than a predefined threshold increase, and the sixth column indicates the decision to be made if, at that time, the blood pressure of the subject has increased more than the predefined threshold increase. This predefined threshold increase is typically (but not necessarily) lower than the threshold increase that is used by control unit 50, after stimulation is completed, to determine whether a test site is a target site for ablation (i.e., a “hot spot”). For example, this threshold increase may be 20-70 percent of the threshold increase that is used by control unit 50 to determine whether a test site is a target site for ablation.

As described hereinabove, for some applications, should electrical contact quality drop below a threshold quality during the application of excitatory current (i.e., before the application of excitatory current is completed), control unit 50 returns to step 226. This is the case for contact quality scenarios H-L which, if identified during stimulation, result in abortion of the stimulation and a return to step 226 (see the fifth and sixth columns for these scenarios).

Attention is drawn to contact quality scenarios B-G, which are marked with asterisks. According to this example, if any of these contact quality scenarios are identified during step 226, control unit 50 does not proceed to step 228 (even if blood pressure was sufficiently stable). This is indicated in the fourth column, in which some scenarios indicate that the catheter should be repositioned in order to improve electrical contact, and in which some scenarios indicate that the catheter should be reconnected or replaced. Nonetheless, if these same contact quality scenarios are identified during step 234 (e.g., during stimulation that was initiated in response to previously-sufficient quality of BP stability and electrical contact), control unit 50 only aborts the stimulation and returns to step 226 if BP has not increased more than a threshold increase during the stimulation (fifth column). However, if blood pressure has increased more than this threshold increase during the stimulation, control unit 50 continues the stimulation even for these contact quality scenarios (sixth column).

It is hypothesized by the inventors that, if sub-threshold electrical contact is identified after a first period of stimulation (e.g., if electrical contact becomes reduced during this first period), if it appears that continued stimulation will nonetheless result in a BP increase that indicates a target site (e.g., “hot spot”), it is advantageous to continue stimulation (i) to avoid non-identification of actual target sites, and/or (ii) to avoid unnecessary rounds of stimulation.

Typically, electrical contact quality is determined throughout the application of excitatory current, and if the electrical contact quality of a particular electrode drops sufficiently, control unit stops driving it to apply the excitatory current. For such applications, the continued stimulation described with reference to FIG. 12 is performed by continuing to drive the remaining electrodes to apply the excitatory current. For example, in scenario D, the continued stimulation would be performed by continuing to drive the two “green” and one “yellow” electrode to apply the excitatory current, while not driving the one “red” electrode.

It is to be noted that the specific scenarios in the lookup table of FIG. 12, and the actions associated with them, are examples, and that (i) other possible scenarios exist (i.e., other combinations of electrode contact qualities), and (ii) the action associated with each scenario may be different to that shown. What should be noted, is that electrical contact quality scenarios exist that, if present during step 226, prevent the initiation of stimulation, but that if present during decision 234, only lead to abortion of stimulation if the response to stimulation is lower than a particular threshold.

Therefore, for some applications, a method comprises, using at least one computer processor:
detecting a first degree of electrical contact between one or more electrodes and tissue of the subject;
only if the first degree of electrical contact is above a contact threshold, initiating driving the electrodes to apply excitatory current to the tissue;
during the application of excitatory current, detecting (i) a second degree of electrical contact between the electrodes and the tissue, and (ii) a blood pressure change since the initiating of the driving of the electrodes;
determining whether (i) the second degree of electrical contact is below the contact threshold and (ii) the detected blood pressure change is greater than a pressure-change threshold; and
in response to determining that (i) the second degree of electrical contact is below the contact threshold and (ii) the detected blood pressure change is greater than the pressure-change threshold, continuing to drive the electrodes to apply excitatory current.

That is, as shown for scenarios B-G, if the BP change is greater than the pressure-change threshold, the computer processor continues to drive the electrodes to apply excitatory current despite the second degree of electrical contact being below the contact threshold.

In step 236, the application of excitatory current is complete, and data representative of the detected value of the response (e.g., the value and/or change in blood pressure) are held (e.g., temporarily) by control unit 50 (step 236). It is to be understood that the scope of the invention includes the held data being (or including) the actual detected value, or being otherwise representative of the detected value. For example, the detected value may be processed (or pre-processed). Purely illustrative examples of such processing include simplification (e.g., rounding), categorization (e.g., binning) and multiplication by a constant or by a function. For some applications, and as shown, the detected value is simplified such that if the detected value is above a threshold (e.g., if a change in blood pressure in response to the excitatory current is greater than a threshold change), GUI 260 indicates that the test site is a target site for subsequent application of ablation energy. For example, and as shown, the term “HOT SPOT” may be used (FIG. 7E).

Subsequently, the operator identifies the test site on the schematic map of the vasculature of the subject (step 238). As described hereinabove, the schematic map may have been previously generated, and is retrieved from memory 58 during hotspot-locating subroutine 84. For some applications, the schematic map may have been generated immediately prior to performing subroutine 84. Schematic map 101 (the generation of which is described hereinabove with respect to FIGS. 3A-J) is used here to illustrate subroutine 84 (e.g., steps 238 and 240 thereof).

Control unit 50 displays schematic map 101 on display 40, (e.g., as part of GUI 260) (FIG. 7F). In step 238, the operator inputs (e.g., using a pointer 272) the location 274 on map 101 that corresponds to the test site in the actual blood vessel of the subject (FIG. 7G). In response to this user-inputted location, control unit 50 assigns the held data to the user-inputted location (e.g., stores the data in association with the user-inputted location) (step 240). In the case of location 274, the test site is a target site (e.g., a hotspot). For some applications, and as shown, if the test site is a target site (e.g., a hot spot), this is indicated on map 101, e.g., by changing the color of user-inputted location 274.

It is hypothesized by the inventors that inputting the location on the map (step 238) subsequently to stimulating and detecting the response to the stimulation advantageously reduces the number of steps in a typical procedure. For example, it may prove difficult to successfully complete stimulation at an initially-chosen target site (e.g., due to low electrical contact quality), and therefore the operator may have to repeatedly move device 30 until good (and stable) electrical contact is made between electrode 34 and the vessel wall. If step 238 were performed before the stimulation and detection, it would be necessary to repeat step 238 each time tool 30 is moved, rather than only once after each successfully completed stimulation.

For some applications, control unit 50 determines ablation energy parameters (e.g., characteristics) that it recommended to be used for each identified target site. This may be performed during subroutine 84 (e.g., responsively to receiving the detected value of the physiological parameter, or the data representative thereof), or at a later time (such as prior to or during ablation subroutine 86). Such ablation energy parameters may include amplitude, duration, frequency, and/or modality of ablation energy.

For some applications, the data representative of the detected value includes the determined ablation energy parameters. That is, for some applications, the determined ablation energy parameters are included in the data that is held and subsequently stored in association with the location on the map. For some such applications, these ablation parameters may be displayed on a parameter indicator 276, e.g., continuously, or in response to selecting or “mouseover” of the corresponding target site (FIG. 7H).

Although not shown in flowchart 220, one or more of the test sites may receive more than one application of excitatory current, with each application having different characteristics. For example, if a low or absent response is detected in response to a first application of excitatory current, a subsequent application of excitatory current, having one or more parameters different from the first application, may be applied (a) having a different (e.g., greater) amplitude, (b) having a different frequency, (c) using a different spacing between electrodes, and/or (d) using a different modality (e.g., monopolar or bipolar). This change of stimulation parameters is represented by step 224 of flowchart 220, and the iterative process is represented by connector 246 leading to step 224.

For some applications, the modification of the parameter(s) of the excitatory current facilitates determination of the depth of the nerve fiber, i.e., the distance of the fiber from the lumen of the blood vessel. For example, at a test site at which a greater amplitude of excitatory current and/or a greater distance between electrodes is required to elicit a response, this may indicate that the nerve fiber(s) of interest are at a greater distance from the lumen of the blood vessel.

For applications in which control unit 50 determines ablation energy parameters for an identified target site, the control unit may do so in response to one or more of the characteristics of the excitatory current that successfully induces a response of sufficient magnitude. For example, control unit 50 may recommend a greater amplitude and/or distance between electrodes when a nerve fiber at a target site is at a greater depth.

Alternatively or additionally, the control unit may determine ablation energy parameters in response to the magnitude and/or speed of the response (and for some applications the control unit may do this even when only a single application of excitatory current is used). For applications in which control unit 50 determines ablation energy parameters for an identified target site, the control unit may display, in an association with the user-inputted location (of the test site), (i) characteristics of the excitatory current that successfully induced a response of sufficient magnitude, and/or (ii) recommended characteristics of ablation energy for subsequent ablation (e.g., on indicator 276), such that a physician may determine, based on this displayed information, what ablation energy characteristics to use. Alternatively, and as described in more detail hereinbelow, control unit 50 may automatically use the recommended parameters, e.g., without displaying them.

Steps 222-240 are typically repeated for a plurality of test sites. FIGS. 7I-K show the same steps as do FIGS. 7E-G, but for a test site at which the response to the applied excitatory current is below the threshold response, thereby identifying the test site as not being a target site for ablation. For example, FIG. 7I shows GUI 260 indicating that the test site is a “COLD SPOT”. FIG. 7J shows map 101 including location 274 that was previously identified as a target site. FIG. 7K shows the operator inputting the location 278 of the test site, and the held data being stored in an association with the user-inputted location (steps 238 and 240). As shown in FIG. 7K, if the test site is not a target site (e.g., is a “cold spot”), this is indicated on map 101, e.g., by changing the color of user-inputted location 278 to a color that is different from locations that are target sites. For some applications, identified “cold spots” are not recorded in association with locations on the map.

As described hereinabove, test sites (i.e., user-inputted locations) are defined as target sites (e.g., “hot spots”) based on detected responses to the excitatory current. For some applications, control unit 50 defines each site in response to the detected response to the application of excitatory current at that site. For example, the suitability of a given site as a target site may be determined solely on the detected response to the application of excitatory current at that site. For some applications, control unit 50 defines the target sites based on the detected response to the application of energy at more than one site. For example, for some applications control unit 50 defines as target sites the test sites whose excitation results in the greatest response (e.g., the top n sites, where n may be an absolute number or a percentage of the total number of text sites).

Therefore, with reference to FIGS. 6 and 7A-K, a method is described of using at least one computer processor to:
(a) detect, using a sensor, a physiological parameter of the subject; and
(b) in response to the detected physiological parameter, determine a value for the parameter (or a change in the value), and store, in association with the user-inputted location, data representative of the value (or the change in the value).

For some applications, these steps (a) and (b) are part of the same method as steps (a), (b) and (c) described with reference to FIGS. 3A-J, 4A-G, and 5.

For some applications, control unit 50 provides a function by which the operator may manually add hot spots and cold spots to locations on the map. That is, for some applications, in response to receiving (i) a user-inputted location and (ii) user-inputted data (such as a hot spot or cold spot designation, or values of the physiological parameter), control unit 50 stores the user-inputted data in association with the user-inputted location.

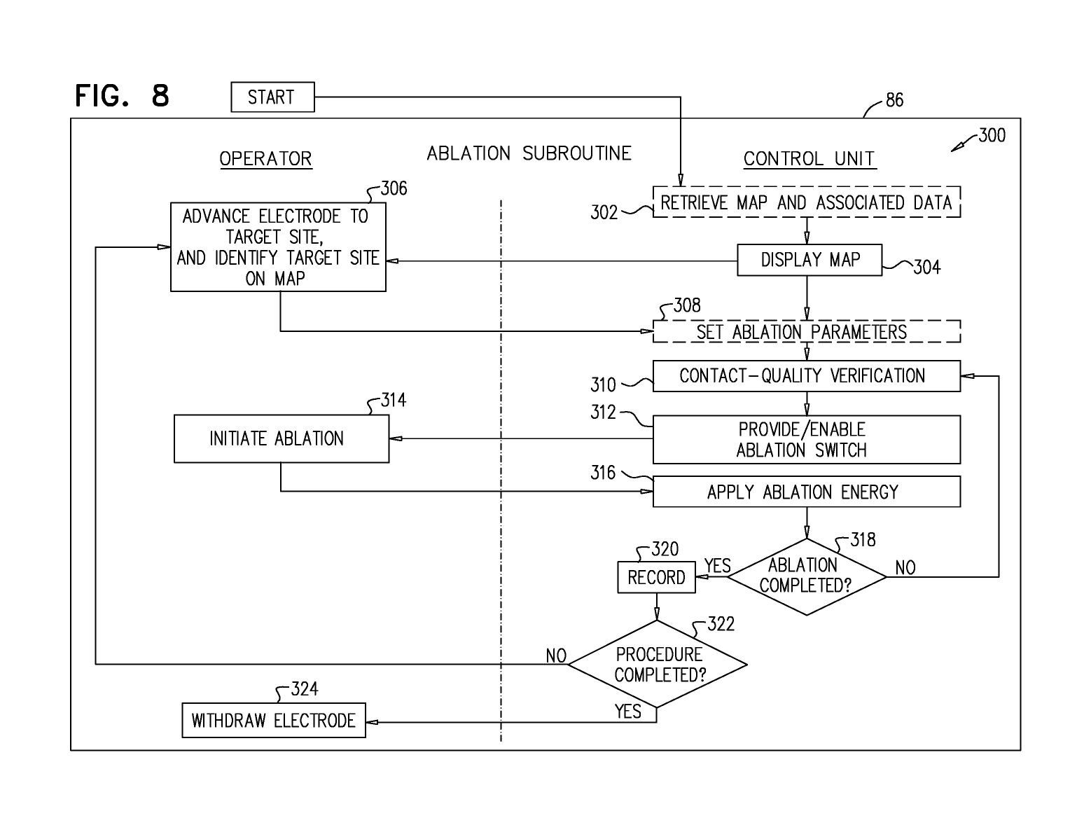

Reference is made to FIG. 8, which is a flowchart 300 showing at least some steps of subroutine 86, in accordance with some applications of the invention. The flowchart is arranged to distinguish between steps performed by the operator, e.g., the physician (on the left side of the flowchart), and those performed by control unit 50 (on the right side of the flowchart).

To begin ablation subroutine 86, if subroutine 84 was performed on a previous occasion, control unit 50 retrieves, from memory 58, the schematic map (e.g., map 101 or 151) for the particular subject and the associated data (i.e., the data associated with user-inputted locations on the map during subroutine 84) (step 302). For applications in which subroutine 86 is performed immediately subsequently to subroutine 84, a discrete step 302 may not be required. Control unit 50 displays the map on display 40, typically including at least some of the associated data; for example, the location of target sites (i.e., “hot spots”) (step 304). For some applications, “cold spots” are not displayed.

The operator advances one or more ablation electrodes to a target site (i.e., a “hot spot”), and identifies that target site by inputting the location of that target site on the displayed map (step 306). For some applications, in response to this input, control unit 50 automatically sets the recommended parameters (e.g., characteristics) of the ablation energy to be used at that site (step 308). As briefly remarked hereinabove, for some such applications, control unit 50 will have previously determined these recommended parameters and stored them associated with the target site during subroutine 84. For some such applications, control unit 50 determines these recommended parameters during step 308 (e.g., in response to stored (and now retrieved) data representative of the response to stimulation during subroutine 84). For some such applications, control unit 50 provides the operator with the opportunity to adjust these recommended parameters. For some such applications, control unit does not provide such an opportunity.

The ablation electrodes are typically disposed at a distal portion of an ablation tool (e.g., comprising a catheter), similarly to the way in which electrodes 34 are disposed on tool 30, mutatis mutandis. Control unit 50 typically interfaces with the ablation tool via catheter interface 54 (or via a different, dedicated ablation catheter interface). For some applications, tool 30 is used to perform ablation subroutine 86.

Subsequently, control unit 50 performs contact-quality verification, e.g., as described for subroutine 84, mutatis mutandis (step 310), and once electrode contact quality is determined to be sufficient, an ablation switch is provided and/or enabled, e.g., as described with respect to stimulation switch 266, mutatis mutandis (step 312). The operator is then able to initiate ablation by operating the ablation switch (step 314). In response, control unit 50 drives the ablation electrode(s) to apply the ablation energy at the target site (step 316).

Typically, contact-quality verification is performed during the application of ablation energy, in a similar way to that described above for the application of excitation current. Typically, if contact quality drops below a threshold, the application of ablation energy is aborted, e.g., and control unit 50 returns to step 310. This is represented by decision 318. For some applications, even if overall contact quality remains above the threshold, if contact quality of a particular electrode drops sufficiently, control unit 50 stops driving that electrode to apply the excitatory current. If the application of ablation energy completes, this fact is typically recorded (i.e., data representative of this fact is stored in an association with the user-inputted location of the target site) (step 320). For some applications, incomplete ablations are also stored.

There is therefore provided a method for use with a subject, the method comprising, using at least one computer processor:

detecting a first degree of electrical contact between a plurality of electrodes and tissue of the subject;

only if the first degree of electrical contact is above a contact threshold, initiating driving the electrodes to apply excitatory current to the tissue (e.g., as shown in the fourth column of FIG. 12);

after the start of the application of excitatory current, detecting (i) a second degree of electrical contact between the electrodes and the tissue, and (ii) a blood pressure change since the initiating of the driving of the electrodes;

determining whether (i) the second degree of electrical contact is below the contact threshold and (ii) the detected blood pressure change is greater than a pressure-change threshold; and

in response to determining that (i) the second degree of electrical contact is below the contact threshold and (ii) the detected blood pressure change is greater than the pressure-change threshold, continuing to drive at least one of the electrodes to apply excitatory current (e.g., as shown in the sixth column of FIG. 12 for scenarios B-G).

The aforementioned steps are repeated for each target site that is to be ablated (represented by decision 322 and the connector back to step 306), and the ablation tool is then withdrawn from the subject (step 324).

Ablation subroutine 86 is described with reference to ablation energy being applied via ablation electrodes. Typically, such ablation energy is provided in the modality of radio-frequency (RF) current. However, it is to be noted that for some applications other ablation modalities are used, mutatis mutandis, including those that are not applied via ablation electrodes. For example, focused ultrasound, cryoablation, and/or chemical ablation may be used, mutatis mutandis.

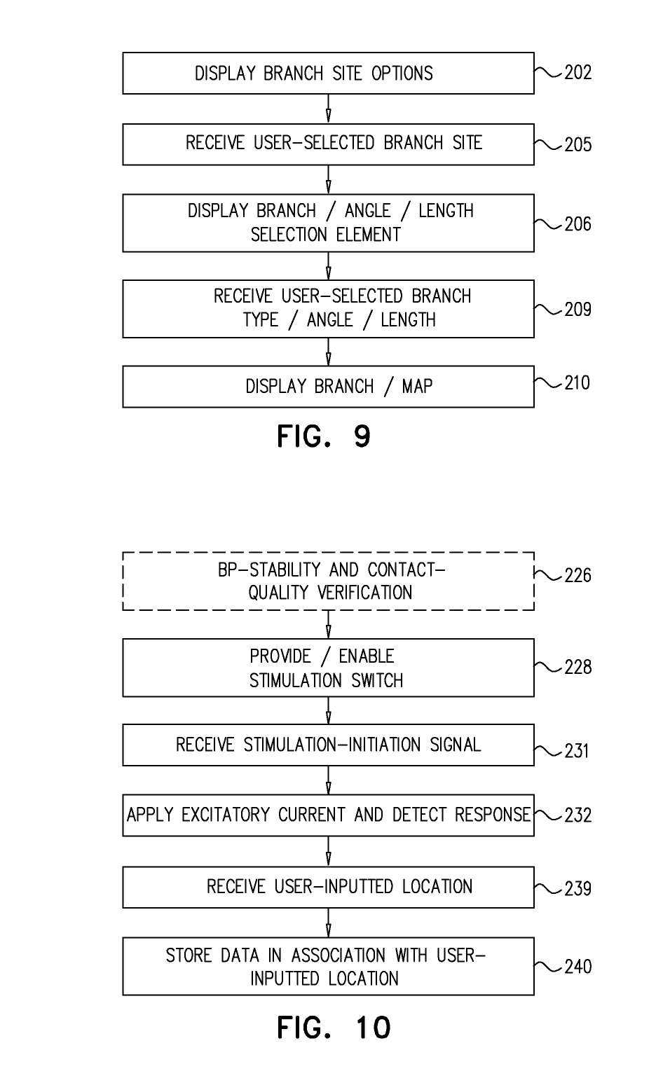

Reference is now made to FIGS. 9-11, which are flowcharts showing at least some steps of respective algorithms performed by control unit 50 (e.g., processor 52 thereof), in accordance with an application of the inventions. FIGS. 5, 6 and 8 are flowcharts of subroutines 82, 84 and 86, respectively, showing steps performed by control unit 50 and steps performed by the operator. FIGS. 9, 10 and 11 are flowcharts of these subroutines, modified to show only steps performed by control unit 50.

FIG. 9 shows at least some steps performed by control unit 50 during subroutine 82, in accordance with some applications of the invention. Steps 202, 206, and 210 are described with reference to FIG. 5. In step 205 control unit 50 receives the user-selected branch site (whereas in the flowchart of FIG. 5, step 204 represents the operator selecting the branch site). Similarly, in step 209 control unit 50 receives the user-selected branch type/angle/length (in contrast to the flowchart of FIG. 5, in which step 208 represents the operator selecting these details).

FIG. 10 shows at least some steps performed by control unit 50 during subroutine 84, in accordance with some applications of the invention. Steps 226, 228, 232, and 240 are described with reference to FIG. 6. In step 231 control unit 50 receives a stimulation-initiation signal provided by the operator (whereas in the flowchart of FIG. 6, step 230 represents the operator initiating the stimulation). Similarly, in step 239 control unit 50 receives the user-inputted location (in contrast to the flowchart of FIG. 6, in which step 238 represents the operator inputting this location).

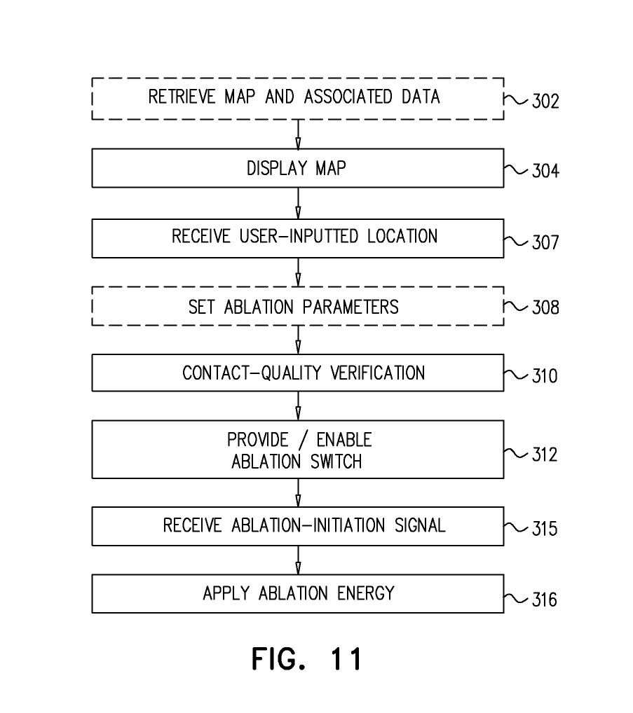

FIG. 11 shows at least some steps performed by control unit 50 during subroutine 86, in accordance with some applications of the invention. Steps 302, 304, 308, 310, 312, and 316 are described with reference to FIG. 8. In step 307, control unit 50 receives the user-inputted location (whereas in the flowchart of FIG. 8, step 306 represents the operator inputting the location). Similarly, in step 315 control unit 50 receives the ablation-initiation signal provided by the operator (whereas in the flowchart of FIG. 8, step 314 represents the operator initiating the ablation).

Conversely, the operator may follow a method comprising:
viewing, on display 40, (i) a schematic representation of vasculature of the subject, and (ii) a plurality of indicators of respective target sites within the vasculature;
by selecting one of the target sites using the system, activating the system to retrieve ablation energy parameters specific for the one of the target sites;
transluminally advancing a tool that includes an electrode at a distal portion thereof, into the vasculature of the subject, such that the electrode becomes disposed at the selected site; and
activating the system to drive the electrode to apply ablation energy using the parameters specific for the selected site.

Applications of the invention described herein can take the form of a computer program product accessible from a computer-usable or computer-readable medium (e.g., a non-transitory computer-readable medium), providing program code for use by or in connection with a computer or any instruction execution system, such as computer processor 52. For the purposes of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor (e.g., computer processor 52) coupled directly or indirectly to memory elements (e.g., memory 58) through a system bus. The memory elements can include local memory employed during actual execution of the program code, bulk storage, and cache memories which provide temporary storage of at least some program code in order to reduce the number of times code must be retrieved from bulk storage during execution.

The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object oriented programming language such as Java, Smalltalk, C++ or the like and conventional procedural programming languages, such as the C programming language or similar programming languages.

It will be understood that blocks of the flowcharts shown in the figures, and combinations of such blocks, can be implemented by computer program instructions. These computer program instructions may be provided to a processor of a general purpose computer, special purpose computer, or other programmable data processing apparatus to produce a machine, such that the instructions, which execute via the processor of the computer (e.g., computer processor 52) or other programmable data processing apparatus, create means for implementing the functions/acts specified in the flowcharts and/or algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the flowchart blocks and algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowcharts and/or algorithms described in the present application.

Computer processor 52 is typically a hardware device programmed with computer program instructions to produce a special purpose computer. For example: (i) when programmed to perform the algorithms described with reference to FIG. 5, computer processor 52 typically acts as a special purpose map-building computer processor, (ii) when programmed to perform the algorithms described with reference to FIG. 6, computer processor 52 typically acts as a special purpose hotspot-locating computer processor, and (iii) when programmed to perform the algorithms described with reference to FIG. 8, computer processor 52 typically acts as a special purpose ablation computer processor.

Typically, the operations described herein that are performed by computer processor 52 transform the physical state of memory 58, which is a real physical article, to have a different magnetic polarity, electrical charge, or the like depending on the technology of the memory that is used.

Techniques described herein may be used in combination with those described in one or more of the following references, which are incorporated herein by reference:

US 2014/0128865 to Gross, filed Feb. 20, 2013;

US 2015/0245867 to Gross, which is a National Phase of WO 2014/068577 to Gross, filed Nov. 3, 2013;

WO 2015/170281 to Gross et al., filed May 7, 2015;

U.S. provisional patent application 62/158,139 to Gross et al., filed May 7, 2015;

U.S. Ser. No. 14/794,737 to Gross et al., filed Jul. 8, 2015;

U.S. Ser. No. 14/795,529 to Gross et al., filed Jul. 9, 2015; and

U.S. Ser. No. 14/972,756 to Gross et al., filed Dec. 17, 2015.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for use with a subject, the method comprising:
using at least one computer processor:
generating a map of vasculature of the subject, by:
displaying, on a display, (i) a schematic representation of a vasculature of the subject that includes a blood vessel of the subject, and (ii) on the schematic representation of the blood vessel, a first plurality of branch-site options;
receiving a user-selected first branch site from the first plurality of branch-site options, and responsively thereto displaying, on the display, (i) a schematic representation of a first branch of the vasculature that branches, at the user-selected first branch site, at an angle from the schematic representation of the blood vessel, and (ii) a second plurality of branch-site options; and
receiving a user-selected second branch site from the second plurality of branch-site options;
at least partially in response to receiving the user-selected second branch site, displaying the map;
detecting, using a sensor, a physiological parameter of the subject; and
in response to the detected physiological parameter:
determining a value for the parameter, and
storing, in association with a location on the map, data representative of the value.

2. The method according to claim 1, wherein the displayed blood vessel, first branch, and second branch collectively define at least part of a vasculature map, and the method further comprises storing the vasculature map on a non-transitory computer-readable medium.

3. The method according to claim 1, further comprising:
displaying an angle-selection input element; and
receiving a user-selected angle via the angle-selection input element,
wherein displaying the schematic representation of the first branch comprises configuring the displayed angle in response to the user-selected angle.

4. The method according to claim 1, further comprising:
displaying an angle-selection input element; and
receiving a user-selected angle via the angle-selection input element,
wherein displaying the schematic representation of the first branch comprises displaying the schematic representation of the first branch extending at the user-selected angle from the schematic representation of the blood vessel.

5. The method according to claim 1, further comprising:
displaying a branch-selection input element comprising a plurality of schematic representations of branch types; and
receiving a user-selected branch type from the plurality of schematic representations of branch types,
wherein displaying the schematic representation of the first branch comprises displaying the user-selected branch type.

6. The method according to claim 1, further comprising:
displaying a length-selection input element; and
receiving a user-selected length via the length-selection input element, wherein displaying the schematic representation of the first branch comprises displaying a length of the schematic illustration of the first branch in response to the user-selected length.

7. The method according to claim 1, further comprising receiving a user-inputted location on the selected branch, wherein storing the data in association with the location comprises automatically storing the data in association with the location in response to receiving the user-inputted location.

8. The method according to claim 7, further comprising, using the at least one computer processor, driving an electrode of an electrode catheter to apply an application of excitatory current, and wherein determining the value for the parameter comprises determining a value of a response of the parameter to the application of excitatory current.

9. The method according to claim 7, further comprising, prior to storing the data in association with the location, holding the data without storing the data in association with the location until the user-inputted location is received, and wherein storing the data in association with the location comprises storing the held data in association with the location in response to receiving the user-inputted location.

10. The method according to claim 7, further comprising, using the at least one computer processor, in response to the value of the detected physiological parameter, defining the location as a target site for subsequent application of ablation energy.

11. The method according to claim 7, further comprising, using the at least one computer processor, in response to the value of the detected physiological parameter, determining at least one ablation energy parameter for subsequent application of ablation energy.

12. The method according to claim 11, wherein the data representative of the value includes the determined ablation energy parameter, and wherein storing, in association with the user-inputted location, the data representative of the value, comprises storing, in association with the user-inputted location, the data that includes the determined ablation energy parameter.

13. Apparatus, for use with a vasculature of a subject, the apparatus comprising:
an intravascular tool, having a distal portion that comprises an electrode and is configured to be transluminally advanced into the vasculature;
a blood pressure sensor;
a display; and
a control unit:
comprising at least one computer processor, and
interfaced with the tool, the blood pressure sensor, and the display, and configured to:
drive the electrode to apply an application of excitatory current, such that if the electrode is within the vasculature, the electrode applies the excitatory current to the vasculature;
automatically detect, using the sensor, a change in blood pressure of the subject induced by the application of the excitatory current;
display, on the display, a schematic representation of the vasculature;
receive a user-inputted location on the schematic representation of the vasculature; and
in response to (i) the user-inputted location, and (ii) the detected change in the physiological parameter, store, in association with the user-inputted location, data representative of the change in blood pressure.

14. The apparatus according to claim 13, wherein the control unit is configured to configure the excitatory current to be an excitatory current having a frequency of 1-300 Hz, and configured to induce action potentials in nerve tissue of the vasculature.

15. The apparatus according to claim 13, wherein the control unit is configured:
to (i) determine a degree of electrical contact between the electrode and the vasculature of the subject, and (ii) use the sensor to determine a blood pressure stability of the subject; and
in response to determining that (i) the degree of electrical contact is above a threshold degree of electrical contact, and (ii) the blood pressure stability is above a threshold degree of blood pressure stability, to drive the electrode to apply the application of excitatory current.

16. The apparatus according to claim 13, wherein the control unit is configured:
to (i) determine a degree of electrical contact between the electrode and the vasculature of the subject, and (ii) use the sensor to determine a blood pressure stability of the subject;
in response to determining that (i) the degree of electrical contact is above a threshold degree of electrical contact, and (ii) the blood pressure stability is above a threshold degree of blood pressure stability, to enable a user-operated switch; and
in response to operation of the switch, to drive the electrode to apply the application of excitatory current.

17-27. (canceled)
